(19) United States
(12) Patent Application Publication
(10) Pub. No.: US 2004/0030502 A1
(43) Pub. Date: Feb. 12, 2004

(54) Process for storing data

(76) Inventor: Andrew Charles Russell Martin, Surrey (GB)

Correspondence Address: DARBY & DARBY P.C., P. O. BOX 5257, NEW YORK, NY, 10150-5257, US

(21) Appl. No.: 10/221,832

(22) PCT Filed: Mar. 14, 2001
(86) PCT No.: PCT/GB01/01123

(30) Foreign Application Priority Data
Mar. 14, 2000 (GB) 0006147.3

(51) Int. Cl. G06F 7/00; G06F 17/30; G06F 19/00; G01N 33/48; G01N 33/50

(52) U.S. Cl. 702/19; 707/3

(57) ABSTRACT

This invention relates to a process for storing data that reduces the amount of computer memory needed to store data in a structured form. The format generated by the method of the invention is readable and easily understandable. Furthermore, the steps described above remove redundancy in a data file, simultaneously reducing its size; the markup of the data is minimal. Accordingly, files may be parsed and transmitted more quickly, and less storage memory is required than for conventional data storage methods. In addition, the method of the present invention provides an efficient, structured file, allowing the incorporation of additional information into the file without requiring a specialist parser to be designed.

This invention relates to a process for storing data. In particular, the invention relates to a process that reduces the amount of computer memory needed to store data in a structured form.

All the references referred to herein are hereby incorporated by reference.

Data arrays are generated in a wide variety of applications and processes. For example, large data arrays are commonly generated in digital imaging and database compilation. It is well recognised that storing large data arrays produces large data files and that such data files can be difficult to manipulate, particularly if the data are stored in an unstructured format. Large, unstructured data files can most notably lead to slow transmission between devices and slow parsing of the data.

To date, numerous methods have been established for storing data arrays. Generally, such storage methods create an associated data file or equivalent in which the data are stored in a file format that is particular to the method of storage. Once the data are stored in a file, the data may be transmitted to a further device, or parsed by a parsing program. The suitability of a file format for fast transmission and parsing of data is dependent on how the data are structured within the file format. An efficiently structured file will lead to improved transmission and parsing of the data. Since the file structure is a direct consequence of the method by which the data are stored, an efficient storage format will correlate with improved transmission and parsing properties for the data itself.

Examples of formats for storing data include eXtended Mark-up Language (XML), Abstract Syntax Notation One (ASN.1) and the macromolecular Crystallographic Information File (mmCIF) format, a variant of the Self-defining Test Archive and Retrieval format (STAR).

XML is becoming an accepted standard for data interchange and has many advantages associated with it. In particular, XML parsers are freely available for C++, Java, JavaScript, Tcl, Python, C and Perl. Additionally, the file script is well structured in a hierarchical, object oriented manner. However, one major disadvantage of XML is that the files become excessively bloated with mark-up language. First, storage requirements are large. Second, this makes the files slow to parse. Third, the files are slow to transmit, for example over a modulator/demodulator (modem) linkage. Furthermore, manual examination of these files is difficult. Since it is useful to be able to examine data files by eye, this is in fact a significant disadvantage.

ASN.1 is the standard adopted by the National Center for Biological Information (NCBI); in a similar way to XML, the format is complex and uses a significant amount of markup. A particular advantage of this standard is that a freely available parser is provided by the NCBI. However, the principal drawback with ASN.1 is the complexity of the format, meaning that methods of implementing a customised format or parser are not in any sense simple.

The mmCIF format consists of data definition blocks followed by simple column-wise data. The advantage of this format is that the large data sections allow relatively fast parsing on freely available parsers. However, adding additional information is not possible without causing the format to stray from the standard. Furthermore, the data in the file are unstructured resulting in a duplication of data elements in the data sections. This naturally leads to excessively large files resulting in slow transmission, less than optimal parsing, and the possibility of errors or conflicts invalidating the data.

One example of a data format that could be immeasurably improved by storing data more efficiently is a Protein Data Bank (PDB) data file. The Protein Data Bank is an archive of experimentally determined three-dimensional structures of biological macromolecules. The archives contain atomic co-ordinates, bibliographic citations and primary and secondary structure information.

In the part of the file that gives details of the atomic co-ordinates for all the atoms in the protein, data are given concerning the protein chain label, the sequence of the residues in the chain and the spatial positions of individual atoms in each residue of the protein. Protein chain lengths can range from a few tens to thousands of residues, meaning that the size of a data file containing information relating to a single protein can potentially be very considerable.

At present, the PDB format is the standard format for storing protein structure data. The format suffers from several disadvantages that can be attributed to the method and file structure by which this format stores data. In particular, the PDB format uses relatively unstructured comment and data regions leading to repetition of data and hence large files. One manifestation of this is that the large files generated are slow to parse and transmit over a network or a modem linkage. Furthermore, the PDB format is inflexible and there is no straightforward way to extend the format to add any additional information.

There is therefore a great need for a data storage method and a file format that is well-structured, that is easily extendable and that is fast to parse and transmit.

### SUMMARY OF THE INVENTION

The present invention provides a process for storing multi-record data in a computer-readable data file, each instance of said data being associated with a plurality of data-fields, wherein said data are listed in columns in a body section of the file, with each column containing data-fields that are associated with the same data-instance and the data-field that is associated with each column is defined in a header section of the file, said process comprising the steps of:

(a) selecting a block of data-instances that share a common value for a particular data-field; and

(b) inserting an append tag defining the common value of said data-field in an append section that precedes the block of data-instances in the body section of the file, the meaning of said append tag being defined in the header section of the file, such that when the data file is read, each of said data-instances in the block inherit this common value.

Such a process alleviates the problems associated with the methods of the prior art. The format generated by the method of the invention is readable and easily understandable. Furthermore, the steps described above remove redundancy in a data file, simultaneously reducing its size; the markup of the data is minimal. Accordingly, files may be parsed and transmitted more quickly, and less storage memory is required than for conventional data storage methods. In addition, the method of the present invention provides an efficient, structured file, allowing the incorporation of additional information into the file without requiring a specialist parser to be designed.

By computer-readable file is meant a file in which data are stored electronically.

Data that are suitable for storage in the process of the invention should constitute multi-record data entries. Each multi-record data entry is associated with a number of data-fields, and in each data-field, each data entry has a specific value. Accordingly, each data entry is associated with one or more fields that describe information relating to the data entry.

For convenience, the examples given herein refer mainly to the storage of protein structure files. However, the skilled reader will readily appreciate that this method of data storage is equally applicable to other types of multiple record data for which each data entry is associated with a plurality of parameters. One example might be information relating to vehicle spare parts contained in a warehouse awaiting distribution. For each vehicle part, information must be stored relating to its serial number, its description, the vehicle for which it is intended, its price, its location in the warehouse and so on. A further example is a food product, each item of which has a certain description, weight, price, sell-by date and so on that will be unique to each batch of products.

The computer-readable data file generated by the process of the invention should contain a “header” section and a “body” section.

The header section contains the Data Type Definition (DTD) for the following body data. The body section contains the data.

In a preferred embodiment of the invention, the DTD is written in the Data Definition Language (DDL) defined herein, although other implementations that embody the same concepts as those described herein are equally applicable to the invention. In this embodiment, the header is valid, well-structured XML.

The DTD defines one or more “data-types”. By “data-type” is meant a defined group of variable data types which constitutes all the information on a given leaf-node object. For example, a protein structure might have an atom data-type (describing the atomic coordinates), a sequence data type (information relating to protein sequence) and an experimental data-type (describing the experimental conditions under which they were determined). A separate data-type and data-block would exist for each of these.

Each data-type has a “data-type-name”. The data-type defines one or more pairs of “labels” and “variable-types”, where a “label” provides the name of a variable within a data-type and a “variable-type” gives the type of the variable within the data-type (such as integer, double, character, and so on). The data-type thus defines labels for each item of data (variable) with an associated “variable-type”. Data-types are defined in the header section.

A particular data-type may also contain “append-types”. An “append-type” is defined as a group of variable data-types which is inherited by leaf-nodes in the object hierarchy. Zero or more append-types may be defined within a particular “data-type”.

In the body section, the data itself is split into separate “data-blocks”. Each data-block reflects a single data-type, which is specified in the header. Zero or one data-block in the body is associated with each data-type. A data-block contains the actual data of the specified type in rows (“data-instances”; see below) and will contain “append-instances” if the data-type specifies “append-types”. In a preferred embodiment, each data-block is enclosed within “DATA” tags.

Each row in a data-block is termed a “data-instance”, which refers to the fundamental entities of which the data array is comprised and is thus the leaf node of an object oriented hierarchy. A data-instance forms a row of data in a “data-block” and consists of delimited “data-fields”, preferably delimited using spaces.

Data-instances represent the leaf-nodes of the object hierarchy. For example, in the case of a protein structure, the term “data-instance” refers to information that is specific for a certain atom type.

Data within a data-block consists of free-format columns of “data-fields”. Each data-instance is associated with a number of different “data-fields” that impart information that is relevant to the context of the data-instance. The term “data-field” refers to an item of data within a “data-instance” or “append-instance”. Each data-field is specified by an associated “label” and “variable-type” in the definition of the “data-type” or “append-type” in the DTD. In the case of a protein structure file, one example of a data-field is the “x co-ordinate” entry whose value indicates the spatial position along the x-axis of a particular atom in space. For a particular data-instance, this x co-ordinate entry is associated with a number of data-fields that impart information relevant to the context of the co-ordinate data, for example, the atom number and identity, the corresponding “y” and “z” coordinates, the residue name and number in which the atom occurs, the chain of the protein to which this residue belongs, and so on. Without all of the relevant information for each atom, the information contained in the x co-ordinate data-field is completely meaningless. In this example however, the chain, residue name and residue number will also be characteristics of other atoms.

In a preferred embodiment, data-fields are separated by whitespace. Should a field itself need to contain whitespace, the whole field is enclosed in inverted commas. An inverted comma may be escaped with a back-slash (\).

By “append-instance” is meant a row of data in a “data-block” which is tagged as append data. Append-instances consist of the append tags which specify the “append-type” and a set of delimited “data-fields”, preferably delimited using spaces.

The size of each subsection of a data-block is restricted by the number of data-instances that share the common data-field value that is defined in an associated append tag. In the case of a protein structure file, each data-instance in a data-block represents an individual atom; atoms represent the leaf-nodes of the hierarchy. All atoms are contained within a particular residue and thus share certain properties such as hydrophobicity and residue accessibility. In this example, there is no smaller section of data for which a common parameter exists that can be used to delimit the section size further.

According to the invention, each data-instance may inherit information from higher levels of the object hierarchy. Higher levels are specified using “APPEND” tags placed within a data-block. Data specified in append tags are termed an “append-instance”. Preferably, the data within the append tags constitute a set of whitespace-separated columns. Each data-instance inherits data from the last-read append-instance of every “append-type” associated with that data type in the DTD. In a preferred embodiment, when the data file is read by a parser program, all inherited data are returned (i.e. the contents of the last-read append-instance of each append-type for the current data-type) with each data-instance. However, another parser could be written where these higher object levels are returned as objects in their own right, rather than being associated with the leaf nodes.

More than one higher level may be specified within the DTD for a given data-type, meaning that a number of levels of append-instance data may be inherited by each data-instance. In this fashion, the number of actual entries in the data sections of a file can be significantly reduced without reducing the data content of the file.

The present invention has thus taken the advantageous aspects of existing format types and discarded the negative aspects that are considered to lead to their limitations. Each file produced by the process of the invention contains data-blocks in which the data are listed. This section is akin to the columns contained within formats such as mmCIF. Data representing values in the same data-field (for example, x co-ordinate; y co-ordinate; z coordinate) are listed in the same columns in the data sections.

In order to reduce the amount of data in the file, the process of the invention removes from the body of the data file, data-fields that have a common value in a number of data-instances. The term “common value” refers to the value of two or more data-fields that are equivalent. The value and meaning of each parameter is defined by an append tag preceding the data section.

To illustrate this more clearly, a protein structure file can be used by way of example. Each “x co-ordinate”, “y co-ordinate” and “z co-ordinate” data-field is associated with one particular atom (a data-instance) that is contained within one particular residue that itself resides in one particular chain of the protein molecule. For each data-instance (atom), there is no need to specify the residue type, residue number or chain type in separate columns when the “x co-ordinate”, “y co-ordinate” and “z co-ordinate” values are presented. According to the process of the invention, this information, which is common to a certain set of atoms, can be presented in a tagged section within the data-block. In this fashion, the volume of data in the data-block of a data file can be significantly decreased without reducing its data content, and redundancy in the file is reduced, removing a source of possible conflicting data.

The same is true of information relating to the physical properties of the residue, such as its hydrophobicity, accessibility and details of its contact with a ligand, if applicable. For each residue, this common information can be removed from the data-instances ascribed to the atoms of that particular residue and can be placed in append tags whose meaning is defined in the header section of the file. In this way, the header significantly reduces the number of bits in the data file, particularly when it is considered that a protein may contain thousands of residues.

The next level up the hierarchy that can generally be made is the definition of the chain to which a certain block of amino acid residues belongs. The append tag containing this data precedes the data-instances for the residues that are contained within that particular chain. When the data file is read by a parser program, these data are inherited (i.e. the contents of the last-read append-instance of each append-type for the current data-type) with each data-instance, until such time as a new chain type append tag is encountered. In this fashion, for data-instances that share the same value for certain data-fields, this information is not repeated for each applicable data-instance.

The append tag refers to a label whose meaning is defined in the DTD section of the file, that is separate from the data-block(s) and that defines the value of one or more data-fields contained in the same file. Conveniently, the tag is a conventional tag as used generally in mark-up languages that are common in the art (such as hyper text mark-up language (HTML), and extensible Mark-up Language (XML).

A further advantage of this process for storing data is that append tags can be defined as required, such that additional information can be appended to a file without interrupting its structure. There is no specific structure with which the files produced by the method of the invention must comply. A parsing program can be designed to read the tags appended to the file such that the presence of a certain tag prompts the program to read the relevant data, whilst the absence of the tag has no adverse consequences. In certain file formats such as that used for PDB files, the incorporation of any additional information to that specified by the file structure is not possible. Although alternative file formats such as XML allow the incorporation of additional tags by virtue of their use of mark-up language, the ascribing of such a tag will merely increase the already excessive amount of mark-up in the file, thus further increasing the time taken to parse and transmit such files. In the case of protein structure files, suitable additional parameters that it may be desirable to insert into a protein structure file include hydrophobicity values, information relating to ligand contact, secondary structure, polymorphism occurrence in the population, accessibility, dimerisation and so on.

The method of the invention requires that entries in the data set that represent the same field and that share a common value are selected. This step in the process is akin to known methods for database normalisation (see Codd, E. F. (1974) “Recent investigations into relational database systems”; Proc IFIP Congress). A number of strategies for selecting entries of common value will be suitable for use in the process of the invention. For each type of data (for example, protein structure information, vehicle parts, food products), there will be different elements of the data that have data-fields of the same value. In the case of protein structure information, these elements are those relating to atom definition, residue number and identity, chain number and so on. In the case of vehicle parts with different serial numbers, each will have a different location in the warehouse and a different price, but many will be intended for the same vehicle model/style/engine-size while multiple vehicles will share the same manufacturer. In the case of a batch of a food product such as a tin of beans, there may be tins that possess the same manufacturer or contents, but have different weights, prices, and sell-by dates. Further examples will be clear to the reader.

This identification step may be manually performed, or may be automated. Different conversion programs will be required depending on the input data. Parsers will be independent of the type of data but may vary depending on the needs of the program reading the data. However, once the general method set out above has been understood, the design of such programs is within the skill of those in the art.

The design of a suitable parser to read data files generated by a process according to the invention is within the ability of the skilled reader. The parser specifically designed by the inventor acts by returning all inherited data (i.e. the contents of the last-read append-instance of each append-type for the current data-type) with each data-instance. When an APPEND tag is reached in the data a flag is set internally which causes a user-defined “callback” routine to be called when the following data-instance is read. This parser allows each data-type to be accessed from the in-memory buffer independently of the order in which these data-types appear in the DTD or in the body of the file. Reading of data-instances within a data-type may be “rewound” such that one can read the data two or more times, if required. In a preferred implementation, all data are read and buffered in memory.

The parser may be designed to read the contents of an append-instance without actually reading the leaf-data, although this feature will only be necessary for specific implementations of the invention.

Another preferred feature of the parser is the ability to add fields to a data type (for example, in the case of a protein structure file, it may be desired to add values such as hydrophobicity values, information relating to ligand contact, secondary structure, polymorphism occurrence in the population, accessibility, dimerisation and so on).

According to a further aspect of the invention, there is provided a data file generated by a process according to any one of the aspects of the invention discussed above. Such data files are herein termed “XMAS” (eXtended Markup with Abstract Syntax) files.

According to a further aspect of the invention, there is provided a computer apparatus adapted to perform a process according to any one of the aspects of the invention described above.

In a preferred embodiment of the invention, said computer apparatus may comprise a processor means incorporating a memory means, means for inputting data and computer software means stored in said computer memory adapted to perform a process according to any one of the aspects of the invention described above and output a computer-readable file.

The invention also provides a computer-based system for storing multi-record data, comprising means for inputting data; means adapted to process said multi-record data according to any one of the aspects of the invention discussed above, and means for outputting said data in a computer-readable data file format. Preferably, said means for processing the data are computer software means. As the skilled reader will appreciate, once the novel and inventive teaching of the invention is appreciated, any number of different computer software means may be designed to implement the teaching of the invention.

The system of the invention may comprise a central processing unit; an input device for inputting requests; an output device; a memory; and at least one bus connecting the central processing unit, the memory, the input device and the output device. The memory should store a module that is configured so that upon receiving a request to store multi-record data, it performs the process steps listed in any one of the aspects of the invention described above.

In the apparatus and systems of these embodiments of the invention, data may be input by downloading the data from a local site such as a memory or disk drive, or alternatively from a remote site accessed over a network such as the internet. The data may be input by keyboard, if required.

The computer-readable file may be output in any convenient format, for example, to a printer, a word processing program, a graphics viewing program or to a screen display device. Other convenient formats will be apparent to the skilled reader.

In a still further embodiment, the invention provides a computer program product for use in conjunction with a computer, said computer program product comprising a computer readable storage medium and a computer program mechanism embedded therein, the computer program mechanism comprising a module that is configured to store multi-record data according to the processes of any one of the aspects of the invention described above.

The invention will now be described by way of example, with particular reference to a method for storing protein structure data and a method for storing data relating to vehicle parts.

### EXAMPLES

Data Definition Language

The Data Definition Language (DDL) for the XMAS format is listed below. Square brackets represent optional items. Alternative items are grouped between parentheses and separated by bars. Variables are presented in curly brackets. An ellipsis represents a repeat. Items brackets in dollar signs are abbreviations which are defined below.

Abbreviations
$vartype$ ::== (int | long | float | double | char length={len})
Data Type Definition
[comment text]
<FORMAT TYPE={type}>

<$vartype$>{variable-name}</$vartype$>
. . .
[
<APPEND TYPE={apptype}>

<$vartype$>{variable-name}</$vartype$>

</APPEND>
]
. . .

</FORMAT>
<FORMAT TYPE={type}>

<freetext/>

</FORMAT>
[ . . . ]
Data
<DATA TYPE={type}>

<{apptype}> {variable} [{variable} . . . ] </{apptype}>
[ . . . ]
{variable} [{variable} . . . ]
[ . . . ]

</DATA>
-or for Free Text-
<DATA TYPE={type}>
Free form textual data which
is spread over many lines
</DATA>

### Example 1

### Protein Structure Files

Typical protein data in a PDB file is shown below.

TABLE 1

| Data | | Atom | | Chain | Residue | X | Y | Z | | B |
|---|---|---|---|---|---|---|---|---|---|---|
| Type | No. | Type | Residue | Label | No. | coord | coord | coord | Occupancy | value |
| ATOM | 1 | N | ASP | L | 1 | 15.566 | −9.825 | 34.260 | 1.00 | 18.90 |
| ATOM | 2 | CA | ASP | L | 1 | 15.797 | −11.159 | 34.929 | 1.00 | 19.10 |
| ATOM | 3 | C | ASP | L | 1 | 14.795 | −12.222 | 34.440 | 1.00 | 18.39 |
| ATOM | 4 | O | ASP | L | 1 | 15.154 | −13.300 | 33.969 | 1.00 | 18.09 |
| ATOM | 5 | CB | ASP | L | 1 | 15.673 | −11.028 | 36.462 | 1.00 | 19.92 |
| ATOM | 6 | CG | ASP | L | 1 | 16.056 | −9.635 | 36.972 | 1.00 | 20.74 |
| ATOM | 7 | OD1 | ASP | L | 1 | 15.349 | −8.650 | 36.620 | 1.00 | 21.22 |
| ATOM | 8 | OD2 | ASP | L | 1 | 17.055 | −9.528 | 37.725 | 1.00 | 20.88 |
| ATOM | 9 | N | ILE | L | 2 | 13.525 | −11.878 | 34.555 | 1.00 | 17.75 |
| ATOM | 10 | CA | ILE | L | 2 | 12.444 | −12.754 | 34.172 | 1.00 | 16.66 |
| ATOM | 11 | C | ILE | L | 2 | 12.391 | −12.861 | 32.655 | 1.00 | 16.01 |
| ATOM | 12 | O | ILE | L | 2 | 12.660 | −11.876 | 31.954 | 1.00 | 15.96 |
| ATOM | 13 | CB | ILE | L | 2 | 11.114 | −12.178 | 34.702 | 1.00 | 16.71 |
| ATOM | 14 | CG1 | ILE | L | 2 | 11.138 | −12.091 | 36.234 | 1.00 | 16.64 |
| ATOM | 15 | CG2 | ILE | L | 2 | 9.935 | −13.011 | 34.222 | 1.00 | 16.68 |
| ATOM | 16 | CD1 | ILE | L | 2 | 11.975 | −10.953 | 36.820 | 1.00 | 16.20 |
| ATOM | 17 | N | VAL | L | 3 | 12.107 | −14.065 | 32.164 | 1.00 | 15.00 |
| ATOM | 18 | CA | VAL | L | 3 | 11.975 | −14.316 | 30.735 | 1.00 | 14.14 |
| ATOM | 19 | C | VAL | L | 3 | 10.483 | −14.174 | 30.425 | 1.00 | 13.67 |
| ATOM | 20 | O | VAL | L | 3 | 9.645 | −14.759 | 31.118 | 1.00 | 13.87 |
| ATOM | 21 | CB | VAL | L | 3 | 12.442 | −15.742 | 30.368 | 1.00 | 13.87 |
| ATOM | 22 | CG1 | VAL | L | 3 | 12.270 | −15.996 | 28.882 | 1.00 | 13.38 |
| ATOM | 23 | CG2 | VAL | L | 3 | 13.886 | −15.938 | 30.770 | 1.00 | 13.74 |

Data type: X-ray, resolution = 2.8, R factor = 0.18, free R = 0.23

The above data comprises an array of data, the data being arranged in columns such that the data in each column relate to the same parameter. The parameters with which each column is associated are shown in a box at the top of each column. Data elements associated with the parameters Data Type, Residue, Chain Label, and Residue Number all contain a plurality of elements that share the same value.

In the above example, it can be seen that the data sets associated with the data-fields Data Type, Chain Label and Occupancy have common values for certain groups of “atom” data-instances.

The data-field Chain label is considered first in this example. The value of the data associated with the data-field Chain Label (labelled CHAIN in the file) is stored with the associated parameter Chain Label (CHAIN) using an append tag that is defined in the header section of the file (the DTD). This tag is assigned to all the data sharing the common value. Accordingly, these data are removed from the columns in the file.

In this example, the same process removes the fields “Data Type” (DATA TYPE) and “Residue Type” (RESIDUE). In principle, it would be possible to remove the parameter OCCUPANCY, but since this is a parameter associated with an atom (rather than a residue or chain), to retain the structured format of the data, there would be no sense in doing this.

The XMAS file structure for the data contained in table 1 is as follows.

Two data types are first defined; experimental, and atoms.

<FORMAT TYPE=experimental>

<char length=8>type</char>
<double>resolution</double>
<double>rfactor</double>
<double>freer</double>

</FORMAT>

The above region defines four variables; type, which is a character string, resolution, rfactor and freer, which are numerical variables.

An atom data type is also defined. Most lines only contain the information that varies with every atom. RESIDUE and CHAIN append records will contain those items that vary by CHAIN or RESIDUE and this data will be appended to the following ATOM data. The parser is programmed to return this information for every line.

<HEADER>
<FORMAT TYPE=atoms>

<int>atnum</int>
<char length=4>atnam</char>
<double>x</double>
<double>y</double>
<double>z</double>
<double>occup</double>
<double>bval</double>
<APPEND TYPE=residue>

<char length=4>resnam</char>
<int>resnum</int>

</APPEND>
<APPEND TYPE=chain>

<char length=1>chain</char>

</APPEND>

</FORMAT>
</HEADER>

After the header section, the body-section contains the data.

First, the experimental data are listed,

<DATA TYPE=experimental>

xray 2.8 0.18 0.23

</DATA>

and then the atom data.

<DATA TYPE=atoms>
<CHAIN> L </CHAIN>
<RESIDUE> ASP 1 </RESIDUE>

| | | | | | | |
|---|---|---|---|---|---|---|
| 1 | N | 15.566 | −9.825 | 34.260 | 1.00 | 18.90 |
| 2 | CA | 15.797 | −11.159 | 34.929 | 1.00 | 19.10 |
| 3 | C | 14.795 | −12.222 | 34.440 | 1.00 | 18.39 |
| 4 | O | 15.154 | −13.300 | 33.969 | 1.00 | 18.09 |
| 5 | CB | 15.673 | −11.028 | 36.462 | 1.00 | 19.92 |
| 6 | CG | 16.056 | −9.635 | 36.972 | 1.00 | 20.74 |
| 7 | OD1 | 15.349 | −8.650 | 36.620 | 1.00 | 21.22 |
| 8 | OD2 | 17.055 | −9.528 | 37.725 | 1.00 | 20.88 |

<RESIDUE> ILE 2 </RESIDUE>

| | | | | | | |
|---|---|---|---|---|---|---|
| 9 | N | 13.525 | −11.878 | 34.555 | 1.00 | 17.75 |
| 10 | CA | 12.444 | −12.754 | 34.172 | 1.00 | 16.66 |
| 11 | C | 12.391 | −12.861 | 32.655 | 1.00 | 16.01 |
| 12 | O | 12.660 | −11.876 | 31.954 | 1.00 | 15.96 |
| 13 | CB | 11.114 | −12.178 | 34.702 | 1.00 | 16.71 |
| 14 | CG1 | 11.138 | −12.091 | 36.234 | 1.00 | 16.64 |
| 15 | CG2 | 9.935 | −13.011 | 34.222 | 1.00 | 16.68 |
| 16 | CD1 | 11.975 | −10.953 | 36.820 | 1.00 | 16.20 |

<RESIDUE> VAL 3 </RESIDUE>

| | | | | | | |
|---|---|---|---|---|---|---|
| 17 | N | 12.107 | −14.065 | 32.164 | 1.00 | 15.00 |
| 18 | CA | 11.975 | −14.316 | 30.735 | 1.00 | 14.14 |
| 19 | C | 10.483 | −14.174 | 30.425 | 1.00 | 13.67 |
| 20 | O | 9.645 | −14.759 | 31.118 | 1.00 | 13.87 |
| 21 | CB | 12.442 | −15.742 | 30.368 | 1.00 | 13.87 |
| 22 | CG1 | 12.270 | −15.996 | 28.882 | 1.00 | 13.38 |
| 23 | CG2 | 13.886 | −15.938 | 30.770 | 1.00 | 13.74 |

</DATA>

The format treats anything outside a <FORMAT> or <DATA> block as a comment. The appended tag regions, that is <RESIDUE> and <CHAIN> in this example, are notionally skipped over by the parser since their data will be appended to the subsequent lines. When they are reached, a flag will be set for the first following data line to indicate that they have been hit. This allows the parsing software to identify the beginning of residues and chains etc. without explicit testing. The data is thus stored in the XMAS format and accordingly may be transmitted, parsed or otherwise manipulated much more easily than when using any of the existing file formats.

### Example 2

### Vehicle Parts

Assume car parts are being stored in a warehouse. Data for each part could be tabulated as follows:

| PartNum | BinLoc | Manufacturer | Model | Style | Year | Engine | Description | Price |
|---|---|---|---|---|---|---|---|---|
| 100567 | B23.679 | Renault | Megane | Coupe | 1997 | 1600 | Carburettor | 127.96 |
| 100583 | B28.324 | Renault | Megane | Coupe | 1997 | 1600 | Cam shaft | 98.21 |
| 101273 | C31.232 | Renault | Clio | Sport | 1994 | 1300 | Gear stick | 53.96 |
| 101275 | C31.231 | Renault | Clio | Classic | 1994 | 1300 | Gear stick | 43.96 |
| 110928 | C92.103 | Vauxhall | Cavalier | L | 1986 | 1600 | Oil filter | 9.50 |
| 110237 | C91.102 | Vauxhall | Cavalier | L | 1986 | 1600 | Water pump | 21.25 |

Where:

PartNum is the part number

BinLoc is the warehouse location

One can identify 3 clear object levels in these data:

Manufacturers [who make models of . . . ]
Cars [which contain . . . ]
Parts

The leaf-nodes of the object hierarchy are the details specific to a given part (i.e. the part number, description, warehouse location and price). Information about the car (i.e. model, style, year and engine size) is a higher level, while information about the manufacturer is another higher level:

| Manufacturer | | Vehicle | | CarPart |
|---|---|---|---|---|
| Manufacturer | -> | Model | -> | PartNum |
| | | Style | | BinLoc |
| | | Year | | Description |
| | | Engine | | Price |

The following XMAS DTD implements this scheme:

<HEADER>
<FORMAT TYPE=carparts>

<int>partnum</int>
<char length=8>binloc</char>
<char length=255>description</char>
<double>price</double>
<APPEND TYPE=vehicle>

<char length=80>model</char>
<char length=16>style</char>
<int>year</int>
<int>engine</int>

</APPEND>
<APPEND TYPE=manufacturer>

<char length=80>manufacturer</char>

</APPEND>

</FORMAT>
</HEADER>

The above data may then be presented as follows (indentation is not necessary but makes the structure of the data easier to follow):

<DATA TYPE=carpart>

<manufacturer>Renault</manufacturer>

<vehicle>Megane Coupe 1997 1600</vehicle>

100567 B23.679 Carburettor 127.96
100583 B28.324 “Cam shaft” 98.21

<vehicle>Clio Sport 1994 1300</vehicle>

101273 C31.232 “Gear stick” 53.96

<vehicle>Clio Classic 1994 1300</vehicle>

101275 C31.231 “Gear stick” 43.96

<manufacturer>Vauxhall</manufacturer>

<vehicle>Cavalier L 1986 1600</vehicle>

110928 C92.103 “Oil filter” 9.50
110237 C91.102 “Water pump” 21.25

</DATA>

1. A process for storing multi-record data in a computer-readable data file, each instance of said data being associated with a plurality of data-fields, wherein said data are listed in columns in a body section of the file, with each column containing data-fields that are associated with the same data-instance and the data-field that is associated with each column is defined in a header section of the file, said process comprising the steps of:
(a) selecting a block of data-instances that share a common value for a particular data-field; and
(b) inserting an append tag defining the common value of said data-field in an append section that precedes the block of data-instances in the body section of the file, the meaning of said append tag being defined in the header section of the file, such that when the data file is read, each of said data-instances in the block inherit this common value.

2. A process according to claim 1, wherein the steps of selecting a block of data-instances and inserting an append tag are repeated for each set of data that represent the same data-field and that share a common value.

3. A process according to claim 2, wherein blocks of data are arranged in a hierarchy with each block inheriting the append tag of the blocks within which it is subsumed.

4. The process of claim 3, wherein in step a), the set of data elements selected as the highest level in the data hierarchy is the set which comprises the greatest number of elements that share a common value.

5. A process according to any one of the preceding claims, wherein said data is protein structure data.

6. The process of claim 5 wherein said protein data is Protein Data Bank (PDB) data.

7. A process according to any one of the preceding claims, wherein groups are selected on the basis of the data-fields relating to data type, chain type, residue type, residue number, hydrophobicity value, information regarding ligand contact, secondary structure, polymorphism occurrence in the population, accessibility and dimerisation.

8. A process according to any one of the preceding claims, which is implemented by a computer.

9. A data file generated by a process according to any one of the preceding claims.

10. A data file according to claim 9, which is an XMAS file.

11. A computer apparatus adapted to perform a process according to any one of claims 1-8.

12. A computer apparatus according to claim 11 comprising a processor means incorporating a memory means, means for inputting data and computer software means stored in said computer memory adapted to perform a process according to any one of claims 1-8 and output a computer-readable file.

13. A computer system for storing multi-record data, comprising means for inputting data; means adapted to process said multi-record data according to any one of claims 1-8, and means for outputting said data in a computer-readable data file format.

14. A computer system according to claim 13, comprising a central processing unit; an input device for inputting requests; an output device; a memory; and at least one bus connecting the central processing unit, the memory, the input device and the output device.

15. A system according to claim 14, wherein a module is stored within said memory that is configured so that upon receiving a request to store multi-record data, it performs the process steps listed in any one of claims 1-8.

16. A computer program product for use in conjunction with a computer, said computer program product comprising a computer readable storage medium and a computer program mechanism embedded therein, the computer program mechanism comprising a module that is configured to store multi-record data according to the processes of any one of claims 1-8.
